(19) United States
(12) Patent Application Publication
(10) Pub. No.: US 2024/0350448 A1
(43) Pub. Date: Oct. 24, 2024

(54) DICHLOROACETIC ACID CONJUGATING DIPHENYL ETHANE COMPOUND, AND PREPARATION METHOD AND APPLICATION THEREOF

(71) Applicant: SHANGHAI ECUSTBIOMEDICINECOMPANY LTD., Shanghai (CN)

(72) Inventors: Fanhong WU, Shanghai (CN); Jinwen HUANG, Shanghai (CN); Kangyan XUE, Shanghai (CN); Hui NIE, Shanghai (CN); Fuli LIU, Shanghai (CN); Hui TANG, Shanghai (CN)

(21) Appl. No.: 18/758,544

(22) Filed: Jun. 28, 2024

Related U.S. Application Data
(63) Continuation of application No. PCT/CN2022/081328, filed on Mar. 17, 2022.

(30) Foreign Application Priority Data
Dec. 31, 2021 (CN) 202111665548.4

(51) Int. Cl. A61K 31/24; A61K 47/54; A61P 35/00

(52) U.S. Cl. CPC A61K 31/24; A61K 47/542; A61P 35/00

(57) ABSTRACT

Described herein is a dichloroacetic acid conjugating diphenyl ethane compound, and a preparation method and an application thereof. The dichloroacetic acid conjugating diphenyl ethane compound has a stable structure, low toxicity, a simple preparation process and a high yield, without necessity to preserve it in a dark place, and has anti-tumor activity against a variety of tumor cells cultured in vitro, and the anti-tumor effect in vivo.

### CROSS-REFERENCE TO RELATED APPLICATIONS

This application is a continuation of International Application No. PCT/CN2022/081328, filed Mar. 17, 2022, which claims priority from CN 202111665548.4, filed Dec. 31, 2021, the entire disclosures of which are incorporated herein by reference in their entireties.

### FIELD OF THE INVENTION

The present invention belongs to the technical field of drug synthesis, in particular relates to a dichloroacetic acid conjugating diphenyl ethane compound, and a preparation method and an application thereof.

### BACKGROUND OF THE INVENTION

Data provided by the World Health Organization (WHO) shows that cancer is still the second leading cause of death in the world, thus, because of a defect in the traditional cancer treatments researchers have focused on targeted tumor drugs. The targeted tumor drugs employ a single compound, which acts on two or more different biological targets to enhance the efficacy of inhibiting tumors and concurrently achieve high selectivity and low toxicity. However, the targeted tumor drugs are different at all aspects of efficacy, for example, some of them have high specificity but their efficacy of inhibiting tumors have yet to improve; in addition, a current way of modification on the basis of an anti-tumor drug in order to obtain a drug with better activity is usually a high-risk project that costs hundreds of millions of dollars, takes a ten-year research and development cycle, and confronts various courses of elimination in terms of drug efficacy, approval and the likes.

In 1956, Warburg, a German chemist, discovered that even under aerobic conditions, cancer cells still tend to go through an aerobic glycolysis process rather than an oxidative phosphorylation process of normal cells. This particular phenomenon is called the “Warburg effect”. As indicated in research, an enzyme, pyruvate dehydrogenase kinase (PDK), is an essential substance for glycolysis, so inhibition of this enzyme reverses the Warburg effect; correspondingly, the dichloroacetic acid (DCA) acts as an inhibitor of PDK. Sodium dichloroacetate is a small molecule compound usually used to cure lactic acidosis for many years. In recent years, researchers have found that DCA can act on an energy metabolism course of tumor cells by inhibiting PDK, so as to promote oxidation and phosphorylation of tumor cells, induce apoptosis, and inhibit tumors growth. By way of continuous research, researchers have developed several kinds of PDK inhibitors containing dichloroacetic acid, such as: VER-246608, 2-Chloro-N-(2-methylphenyl) propanamide, and Mitaplatin. However, some problems exist in the clinical application of DCA, for example, the efficacy of DCA is not high in clinical trials, and some patients who use the drug will have symptoms such as pain and numbness, so China has not legitimated this DCA drug for sale. Therefore, it is necessary to seek a novel DCA-derived drug candidate compound with high anti-tumor activity and low toxicity.

### SUMMARY OF THE INVENTION

The technical problem to be solved by the present invention consists in providing a novel compound with anti-tumor activity, and a preparation method and an application thereof.

Diphenyl ethane compounds, such as erianin, are a kind of tubulin-targeting drugs with good anti-tumor activity. Dichloroacetic acid compounds have the activity of PDK inhibitors, but their efficacy is not high in clinical trials; in addition, they may cause pain, numbness or toxic side effects to some patients. In this research, a modification is made on the structure of dichloroacetic acid, so as to improve the anti-tumor activity and reduce the toxic side effects under the premise of functioning as a target. In the previous research, researchers had tried to use many other compound groups (including natural products) to modify or transform the dichloroacetic acid and its derivatives, but got nonideal results, including the problems such as instability of a compound, too short half-lives, low solubility, too strong toxicity, poor anti-tumor activity, too long metabolic time, high costs for preparation, and too severe process conditions. The present invention reveals that the diphenyl ethane compounds such as erianin and the likes conjugated with the dichloroacetic acid in a certain way have good anti-tumor activity and low toxicity. Therefore, the present invention is expected to obtain anti-tumor active compounds with multiple target points by combining a natural product, the diphenyl ethane compounds, with an energy targeting drug, the sodium dichloroacetate.

The present invention provides a dichloroacetic acid conjugating diphenyl ethane compound, the structure of which accords with a general formula (I) as follows

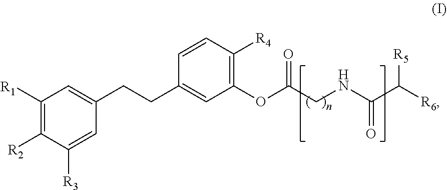

Where, R₁, R₂ and R₃ are independently selected from any one of —OH, —Ome and H, R₄ is selected from any one of —OMe and —OEt, R₅ and R₆ are selected from a chlorine atom or a fluorine atom, m is 0 or 1, and n may be 1, 2, 3, 4, 6, 7, 8, 11, or 12.

Preferably, R₄ is selected from —OMe or —OEt.

The present invention also provides a pharmaceutical composition, the active ingredient of which is the aforementioned dichloroacetic acid conjugating diphenyl ethane compound.

Preferably, the pharmaceutical composition consists of an effective dosage of the aforementioned dichloroacetic acid conjugating diphenyl ethane compound and a pharmaceutically acceptable carrier.

Preferably, its pharmaceutical form is selected from, but not limited to, lyophilized powders, powders, injections, liposomes, emulsions, microcapsules, suspensions or solutions, whichever are administered intravenously; granules, tablets, capsules, or syrups, whichever are administered orally; or suppositories.

Preferably, the pharmaceutical composition is a drug that inhibits proliferation, growth or damage of tumor cells, and eliminates tumor cells.

Preferably, the pharmaceutical composition is applicable to a human being or an animal. The animal is selected from, but not limited to, monkeys, rats, mice, rabbits, cats, dogs, cows, horses, sheep or pigs.

In another aspect, the present invention also provides a preparation method of the aforementioned dichloroacetic acid conjugating diphenyl ethane compound. At the existence of —OH in place of more than one of R₁, R₂ and R₃, its synthetic route is as follows:

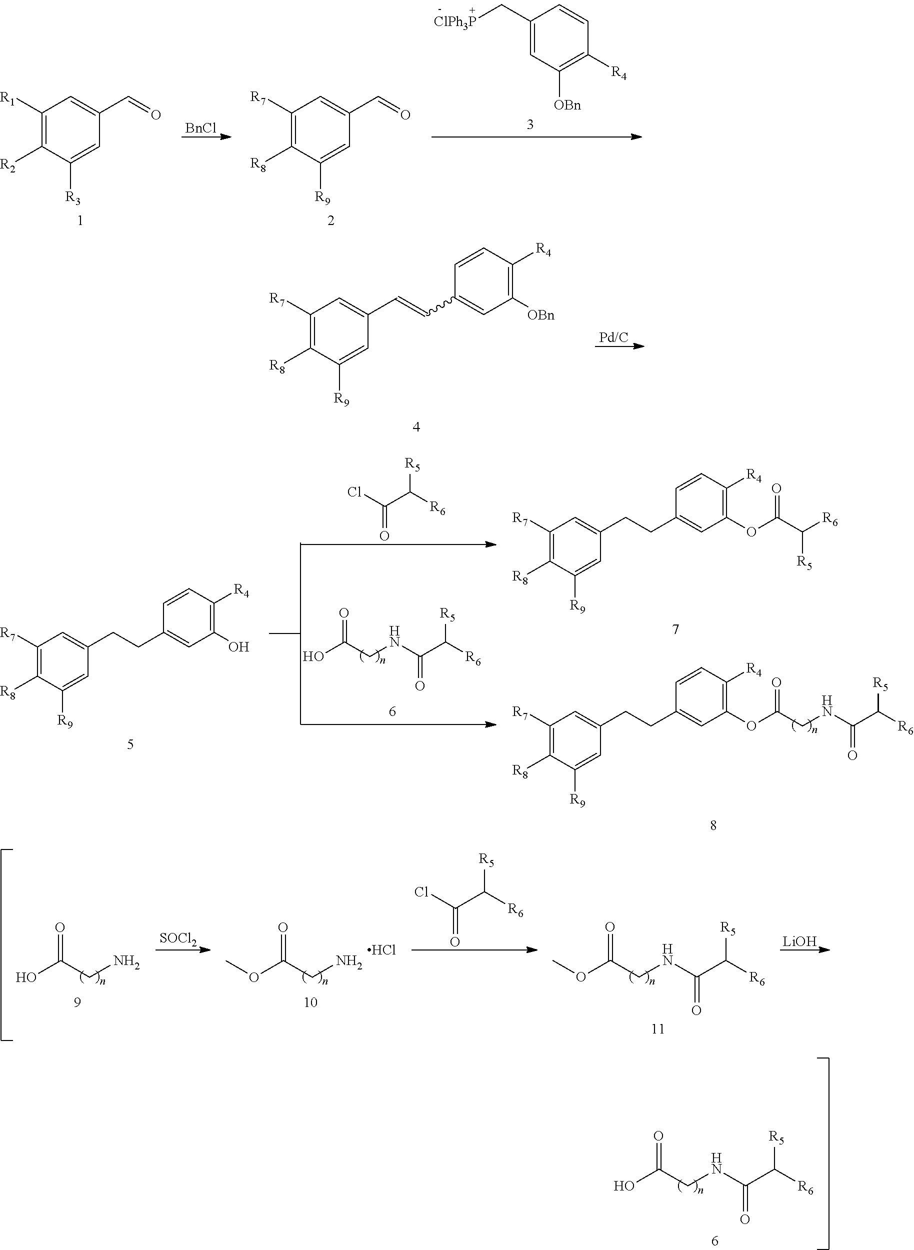

The specific steps are as follows.

(1) At the existence of —OH in place of more than one of R₁, R₂ and R₃, firstly, protect a hydroxyl group in

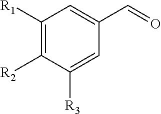

with benzyl chloride to obtain a corresponding benzyl-protected product.

(2) Make the benzyl-protected product and a triphenyl phosphorus ylide

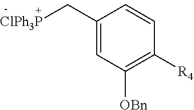

execute a Wittig reaction to obtain a benzyl-protected stilbene compound.

(3) Reduce and deprotect the benzyl-protected stilbene compound to obtain a corresponding diphenyl ethane compound.

(4) In the case that the dichloroacetic acid is directly conjugated to the diphenyl ethane compound, make

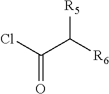

combine with the corresponding diphenyl ethane compound through a condensation reaction to obtain a series of dichloroacetic acid conjugating diphenyl ethane compounds.

In the aforementioned preparation method, at the absence of —OH in place of any one of R₁, R₂ and R₃, omit the step of protection with benzyl chloride, directly execute a Wittig reaction with

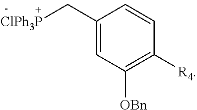

In the aforementioned preparation method, in the case that the dichloroacetic acid is combined with the diphenyl ethane by way of an amino acid having a chain different in length, the method includes the following steps.

(1) Make an amino acid having a chain different in length

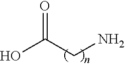

(n=1, 2, 3, 4, 6, 7, 8, 11, 12) react with thionyl chloride to obtain a corresponding amino acid methyl ester hydrochloride

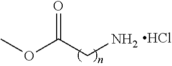

(n=1, 2, 3, 4, 6, 7, 8, 11, 12).

(2) Combine the amino acid methyl ester hydrochloride having a chain different in length with

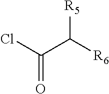

through a condensation reaction to obtain a compound

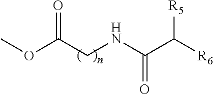

(n=1, 2, 3, 4, 6, 7, 8, 11, 12).

(3) Hydrolyze and demethylate the compound

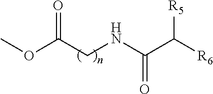

to obtain a corresponding acid

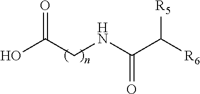

(n=1, 2, 3, 4, 6, 7, 8, 11, 12).

(4) Combine

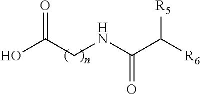

(n=1, 2, 3, 4, 6, 7, 8, 11, 12) with the corresponding diphenyl ethane compound through a condensation reaction to obtain a series of dichloroacetic acid conjugating diphenyl ethane compounds.

In another aspect, the present invention provides an application of the dichloroacetic acid conjugating diphenyl ethane compound in the manufacture of the drugs capable of resisting various tumors, which mainly include a lung cancer, a non-small cell lung cancer, a liver cancer, a pancreatic cancer, a stomach cancer, a bone cancer, an esophageal cancer, a breast cancer, a prostate cancer, a testicular cancer, a colon cancer, an ovarian cancer, a bladder cancer, a cervical cancer, a melanoma, a squamous cell carcinoma, a basal cell carcinoma, an adenocarcinoma, a sweat gland carcinoma, a sebaceous carcinoma, a papillary carcinoma, a papillary adenocarcinoma, a cystic adenocarcinoma, a cystic carcinoma, a medullary carcinoma, a bronchial cancer, a bone cell carcinoma, an epithelial carcinoma, a cholangiocarcinoma, a choriocarcinoma, an embryonic carcinoma, a seminoma, a Wilms cancer, a glial cell carcinoma, an astrocytoma, a medulloblastoma, a craniopharyngioma, an ependymoma, a pineal tumor, a hemblastoma, a vocal cord neuroma, meningioma, a neuroblastoma, an optic neuroblastoma, a retinoblastoma, a neurofibroma, a fibrosarcoma, a fibroblastoma, a fibroma, a fibroadenoma, a fibrochondoma, a fibrocystoma, a fibromyxoma, a fibro-osteoma, a fibromyxosarcoma, a fibropapilloma, a myxosarcoma, a myxocystoma, a myxochondroma, a myxochondrosarcoma, a myxochondrofibrosarcoma, a myxoadenoma, a myxoblastoma, a liposarcoma, a lipoma, a lipoadenoma, a lipoblastoma, a lipochondroma, a lipofibroma, a lipohemangioma, a myxolilipoma, a chondrosarcoma, a chondroma, a chondromyoma, a chordoma, a chorionoma, a chorioadenoma, a trophoblastoma, a chorioblastoma, an osteosarcoma, an osteoblastoma, an osteocardrofibroma, an osteochondrosarcoma, an osteochondroma, an osteocystoma, an osteodentoma, an osteofibroma, an osteofibrosarcoma, an angiosarcoma, an angioma, an angiolipoma, an angiochondroma, an angioblastoma, an angiokeratinoma, an angioglioma, an angioendothelioma, an angiofibroma, an angiomyoma, an angiolipoma, an angiolymphangioma, an angiolipomyoma, an angiomyoneuroma, an angiomyxoma, an angioreticuloendothelioma, a lymphangiosarcoma, a lymphogranuloma, a lymphangioma, a lymphoma, a lymphomyxoma, a lymphosarcoma, a lymphangiofibroma, lymphocytoma, a lymphoid epithelioma, a lymphoblastoma, an endothelioma, an endothelioblastoma, a synovioma, a synoviosarcoma, a mesothelioma, a syndesmoma, a Ewing tumor, a leiomyoma, a leiomyosarcoma, a leiomyoma, a leiomyofibroma, a rhabdomyoma, a rhabdomyosarcoma, a rhabdomyomyxoma, an acute lymphoid leukemia, an acute myeloid leukemia, a chronic cell nosohemia, a polycythemia, a lymphoma, and a multiple myeloma.

The present invention relates to a therapeutic use of the dichloroacetic acid conjugating diphenyl ethane compound. The therapeutic use includes tumors (cancers) or diseases due to abnormal cell proliferation.

Preferably, the anti-tumor drug resists tumors or neoplasms derived from one or more of a gastric cancer cell, a lung cancer cell, a cervical cancer cell, a bowel cancer cell, a breast cancer cell and a liver cancer cell.

Preferably, the anti-tumor drug includes a drug or a reagent that alleviates or inhibits tumor cell proliferation.

Preferably, the dichloroacetic acid conjugating diphenyl ethane compound is added to a culture environment of the tumor cells cultured in vitro, so as to make it possible to control the tumor cells in number.

Preferably, the concentrations of the dichloroacetic acid conjugating diphenyl ethane compound in a cell culture medium range from 0.1 to 10 μM (mol/L).

The present invention also provides a reagent for inhibiting proliferation of a cell, containing the dichloroacetic acid conjugating diphenyl ethane compound. Preferably, the cell is a tumor cell cultured in vitro.

The test results indicate that the dichloroacetic acid conjugating diphenyl ethane compound of the present invention have anti-tumor activity equivalent to or better than that of erianin or sorafenib.

The erianin is expressed as 2-Methoxy-5-[2-(3,4,5-trimethoxyphenyl) ethyl] benzol, its CAS number is 95041-90-0, and its chemical formula is C₁₈H₂₂O₅. The erianin is extracted from a whole plant of orchid, Dendrobium, it is a Chinese herbal monomer having an anti-tumor effect; although there is still some controversy about its anti-tumor effect, but it has been confirmed that it has a good effect of inhibiting proliferation of some tumor cells.

The sorafenib is a novel multi-targeted oral drug used to cure tumors, especially to cure the gastrointestinal stromal tumors and the metastatic renal cell carcinoma that do not respond to a standard therapy or cannot tolerate it. It can selectively target receptors of certain proteins and supposed to act as a molecular switch during tumor growth. It has obtained the “fast track” approval granted by the FDA for the aforementioned therapeutic use in the United States, and has been commonly used as a clinical anti-tumor drug in China.

The drug used to cure tumors provided by the present invention contains an effective dosage of a dichloroacetic acid conjugating diphenyl ethane compound or a dichloroacetic acid conjugating diphenyl ethane compound, and a pharmaceutically acceptable carrier.

In the present invention, LD is an English abbreviation for lethal dosage, which refers to the minimum dosage required for a substance that can cause a death of an organism. In the light of different dosages of drugs and different mortality rates between subjects, LD can usually be classified into different dosages such as a half lethal dosage, an absolute lethal dosage, a minimum lethal dosage and a maximum tolerated dosage.

The IC₅₀ used in the present invention is the half inhibition concentration (or half inhibition rate), which refers to a concentration of the inhibitor inhibiting the “reaction” by half. In terms of anti-tumor, it can be understood as the concentration of the drug used to halve the number of tumor cells.

The term “therapeutic effective dosage” or “effective dosage” as used in the present invention refers to a dosage of the dichloroacetic acid conjugating diphenyl ethane compound that is non-toxic but sufficient to achieve a desired biological, therapeutic and/or prophylactic result. The result may be a reduction and/or alleviation of an omen, a symptom or a cause of a disease, or any other desired alterations of a biological system. For example, the “effective dosage” for a therapeutic use is a quantity of the dichloroacetic acid conjugating diphenyl ethane compound itself required to present a significant improvement of diseases clinically as disclosed herein, or a quantity of the composition containing the dichloroacetic acid conjugating diphenyl ethane compound. The effective dosage appropriate to any individual case may be determined by a person skilled in the art through a routine test. Therefore, the effective dosage of the dichloroacetic acid conjugating diphenyl ethane compound used in the present invention refers to a quantity that can achieve an effective oral use for the dichloroacetic acid conjugating diphenyl ethane compound.

The term “pharmaceutically acceptable” means that a substance is not biologically or otherwise undesirable, that is, the substance can be administered to an individual without causing undesirable biological effects or without interacting in a harmful way with any component of the composition containing the substance.

The term “cure” and its grammatical synonyms include achieving a therapeutic effect and/or a preventive effect. The therapeutic effect refers to alleviation or cradication of a basic symptom being cured. The therapeutic effect is also achieved through eradication or alleviation of one or more of physiological symptoms associated with the basic symptom, so that a patient is observed to have an improvement, despite the fact that the patient may still be afflicted by the basic symptom. The preventive effect may be used for applying the method of the present invention on the patients diagnosed clinically with a tumor, or on patients who have one or more physiologic symptoms as reported despite no diagnosis about tumors, or used for the composition of the present invention administered to these patients.

As used herein, the term “pharmaceutically acceptable carrier” refers to a carrier used for treatment, namely administration, including a variety of excipients and diluents. The term refers to such drug carriers that are not essential active ingredients in themselves and do not have excessive side effects after administration. The suitable carrier refers to a carrier acceptable pharmaceutically and pharmacologically, which may include liquids such as water, saline, glycerol and Tween, as well known to a person skilled in the art. In addition, auxiliary substances such as a disintegrant, a wetting agent, an emulsifier and a pH buffer exist in these carriers.

The drug used to cure tumors provided by the present invention can be made into various dosage forms in the light of different ways of administration. These dosage forms can be administered orally, topically (skin patches, subcutaneous implants), parenterally, such as subcutaneously and intramuscularly, or with the help of an explant. Among them, it is preferable to administrate drugs orally for curing tumors.

In the present invention, the dichloroacetic acid conjugating diphenyl ethane compound is administered to rats in a safe and effective dosage in the case that it is used for a therapeutic use or as a drug. The safe and effective dosage for curing tumors is usually at least about 80 mg/kg by body weight, and not more than 100 mg/kg by body weight in most cases, preferably about 40 to 80 mg/kg by body weight. Of course, taking a human being as an example, the specific dosage is determined in consideration of the factors such as patient's age and weight, his/her natural health status and nutritional status, compound activity strength, administration and metabolic rates, which are all within the knowledge and skill scope of a person skilled in the art, so it is unnecessary to expand them herein.

Prior to administration, the dichloroacetic acid conjugating diphenyl ethane compound may be mixed with sodium carboxymethylcellulose, normal saline, or any other suitable solutions known to a person skilled in the art. The dosage form of the dichloroacetic acid conjugating diphenyl ethane compound may be administered in the form of solid (freeze-dried) or liquid as required.

In the present invention, a representative drug of energy targeting PDK (phosphatidylinositol-dependent kinase) inhibitors, sodium dichloroacetate, is combined with a VEGFR2 targeting diphenyl ethane compound. In the ethoxy diphenyl ethane compound of the present invention, two benzene rings are connected with each other through a single bond, such structure greatly enhances stability of drugs and reduces toxicity simultaneously; its preparation process is more simple, without necessity to separate it by column chromatography, causing a significant increase in process yield and a great decrease in loss of raw materials as well as process cost of unit synthesis; it enhances stability of drugs without necessity to preserve the drugs in a dark place, so that it brings with the preservation and practical application great convenience. The ethoxy diphenyl ethane compound of the present invention has obvious anti-tumor activity against a variety of tumor cells cultured in vitro, and can achieve or even exceed the anti-tumor effect of erianin or sonafenib.

### DETAILED DESCRIPTION OF SOME EMBODIMENTS

The present invention discloses a dichloroacetic acid conjugating diphenyl ethane compound having a structure according with the general formula (I), and a preparation method and an application thereof. The dichloroacetic acid conjugating diphenyl ethane compound of the present invention is conjugated with dichloroacetic acid at the 4′ position of a B aromatic ring of the diphenyl ethane, and the 3′, 4′, and 5′ positions of a ring of the diphenyl ethane are concurrently substituted by a methoxy group, a hydroxyl group or a fluorine atom respectively; the 3′ position of the B ring is substituted by a methoxy group, an ethoxy group, a trifluoromethyl group, a difluoro methyl group and a difluoroethyl group; the chlorine atom of dichloroacetic acid at the 4′ position of the B ring is substituted by a fluorine atom or a bromine atom. In addition, the dichloroacetic acid is combined with the diphenyl ethane by way of an amino acid having a chain different in length, so as to obtain a compound with good anti-tumor activity.

We shall further describe the technical solution of the present invention in combination with the preferred examples as follows.

### Example 1

### Preparing 2-methoxy-5-(3,4,5-trimethoxyphenylethyl) phenyl 2,2-dichloroacetate (Abbreviated as 7a)

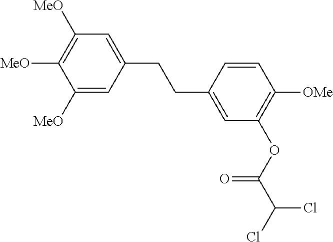 [SMILES: COc1ccc(CCc2cc(OC)c(OC)c(OC)c2)cc1OC(=O)C(Cl)Cl]

Add 10 g (19.05 mmol) of 3-benzyloxy-4-methoxybenzylbenzylchloride triphenyl phosphate and 50 mL of anhydrous tetrahydrofuran into a dried 100 mL three-mouth flask equipped with a thermometer under the protection of nitrogen, then put the flask into an ice salt bath, after the flask is cooled to 0° C., slowly add 3.22 g (28.57 mmol) of potassium tert-butoxide into the flask, and stirring continuously for 30 min. Next, slowly add a tetrahydrofuran solution (10 mL) of 3,4,5-trimethoxybenzaldehyde dropwise for about 20 min, then slowly heat the reaction device to room temperature and continue the reaction for 4 hours. Shut off the reaction device, remove the tetrahydrofuran by means of a rotary evaporation, add 60 mL of water and 100 mL of ethyl acetate to extract out an ethyl acetate layer, then use 60 mL×2 ethyl acetate to get an extract from the water layer to form another ethyl acetate layer; merge the two ethyl acetate layers into one, then dry it by means of anhydrous sodium sulfate. Remove the solvent through reduced pressure distillation to obtain a yellow solid, which is separated by flash column chromatography to obtain 5.89 g of cis-trans products. Then mix the products with palladium carbon, then add the mixture into 24 mL of ethanol, next perform a hydrogen substitution three times, and keeping reacting and stirring for 6 hours. Perform a suction filtration on the palladium carbon by means of diatomite, then obtain 3.21 g of products 5a through separation by flash paper chromatography. Take 500 mg of erianin, then add 8 mL of dry dichloromethane and 0.327 mL of triethylamine into it, and place the solution in an ice salt bath, then stirring it for 10 min. Then slowly add 0.166 mL of dichloroacetyl chloride dropwise and continue reacting for 1.5 hours. After finishing the reaction, add 20 ml of water for quenching, then add 40 mL of dichloromethane to extract out an organic phase, and get an extract from the water layer by means of 20×2 dichloromethane. Merge the organic phases into one, then dry it by means of anhydrous sodium sulfate, next obtain a yellow oily product through reduced pressure distillation, finally obtain 586 mg of white solids with a yield of 86.91% through separation by flash paper chromatography.

1H NMR (400 MHZ, CDCl₃) δ 7.01 (d, J=8.3 Hz, 1H), 6.93 (s, 1H), 6.89 (d, J=8.4 Hz, 1H), 6.33 (s, 2H), 6.19 (s, 1H), 3.81 (d, J=5.0 Hz, 12H), 2.84 (s, 4H); ¹³C NMR (101 MHz, CDCl₃) δ 162.68, 153.15, 149.04, 138.93, 137.10, 136.35, 134.54, 127.77, 121.99, 112.72, 105.53, 77.33, 64.09, 60.92, 56.19, 56.12, 38.26, 36.98.

The above result indicates that the 2-methoxy-5-(3,4,5-trimethoxyphenylyl) phenyl 2,2-dichloroacetate has been successfully prepared by this method.

### Example 2

### Preparing 2-ethoxy-5-(3,4,5-trimethoxyphenylyl) phenyl2,2-dichloroacetate (7b)

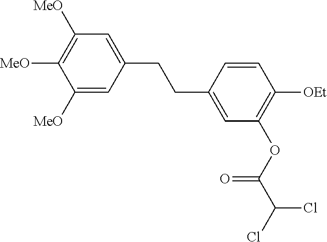 [SMILES: CCOc1ccc(CCc2cc(OC)c(OC)c(OC)c2)cc1OC(=O)C(Cl)Cl]

The synthesis method is as same as that of 7a, finally substitute 3-methoxy-4-benzyloxybenzylchlorotriphenylphosphate with 3-benzyloxy-4-ethoxybenzylbenzylchlorotriphenylphosphate to obtain 526 mg of yellow oily products 7b with a yield of 78.88%.

¹H NMR (400 MHZ, CDCl₃) δ 6.98 (dd, J=8.3, 1.8 Hz, 1H), 6.92 (d, J=1.8 Hz, 1H), 6.88 (d, J=8.3 Hz, 1H), 6.33 (s, 2H), 6.19 (s, 1H), 4.03 (q, J=7.0 Hz, 2H), 3.82 (d, J=5.3 Hz, 9H), 2.84 (s, 4H), 1.37 (t, J=7.0 Hz, 3H); ¹³C NMR (101 MHZ, CDCl₃) δ 162.69, 153.11, 148.35, 137.23, 134.41, 127.71, 121.92, 113.71, 105.58, 64.77, 64.08, 60.95, 56.14, 38.25, 37.00.

### Example 3

### Preparing 2-methoxy-4-(3,4,5-trimethoxyphenyletyl) phenyl 2,2-dichloroacetate (7c)

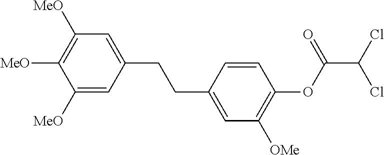 [SMILES: COc1cc(CCc2cc(OC)c(OC)c(OC)c2)ccc1OC(=O)C(Cl)Cl]

The synthesis method is as same as that of 7a, finally substitute 3-benzyloxy4-methoxy-benzylchlorotriphenylphosphate with 3-methoxy-4-benzyloxybenzylchlorotriphenylphosphate to obtain 565 mg of yellow oily products 7c with a yield of 83.83%.

1H NMR (400 MHZ, CDCl₃) δ 7.07 (d, J=7.5 Hz, 1H), 6.75 (dd, J=7.5, 1.4 Hz, 1H), 6.71 (t, J=1.2 Hz, 1H), 6.47 (s, 2H), 6.27 (s, 1H), 3.89 (s, 2H), 3.83 (s, 6H), 2.84 (s, 4H);¹³C NMR (101 MHz, CDCl₃) δ 165.17, 153.21, 153.19, 150.72, 150.72, 150.67, 150.66, 141.99, 141.97, 141.94, 141.92, 136.80, 136.75, 136.71, 136.31, 135.64, 135.59, 122.06, 122.04, 122.02, 122.00, 121.98, 121.26, 121.20, 121.18, 121.15, 113.50, 113.48, 113.46, 113.44, 113.41, 105.94, 105.92, 105.89, 105.87, 105.84, 63.17, 60.79, 56.12, 56.05, 37.64, 37.62, 37.61, 37.59, 37.55, 37.02, 36.99, 36.96, 36.94.

### Example 4

### Preparing 3-(3,4,5-trimethoxyphenylethyl) phenyl2,2-dichloroacetate (7d)

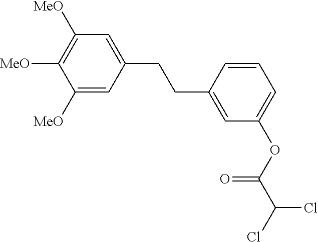 [SMILES: COc1cc(CCc2cccc(OC(=O)C(Cl)Cl)c2)cc(OC)c1OC]

The synthesis method is as same as that of 7a, finally substitute3-benzyloxy4-methoxy-benzylchlorotriphenylphosphate with 3-benzyloxybenzylbenzylchloride triphenyl phosphate to obtain 506 mg of yellow oily products 7d with a yield of 73.12%.

1H NMR (400 MHZ, CDCl₃) δ 7.15 (dt, J=7.4, 1.0 Hz, 1H), 7.09-7.03 (m, 1H), 6.48 (d, J=1.1 Hz, 1H), 3.82 (d, J=2.7 Hz, 4H), 2.89 (m, 2H); ¹³C NMR (101 MHZ, CDCl₃) δ 163.64, 153.10, 150.70, 150.68, 150.64, 150.62, 136.71, 136.38, 136.36, 134.59, 134.56, 134.53, 130.44, 130.41, 130.38, 121.74, 121.73, 121.72, 121.71, 106.55, 106.52, 63.07, 60.80, 56.12, 38.09, 38.06, 38.04, 37.66, 37.63, 37.62, 37.60.

### Example 5

### Preparing 4-(3,4,5-trimethoxyphenylethyl) phenyl2,2-dichloroacetate (7e)

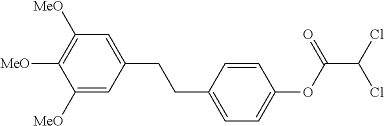 [SMILES: COc1cc(CCc2ccc(OC(=O)C(Cl)Cl)cc2)cc(OC)c1OC]

The synthesis method is as same as that of 7a, finally substitute 3-benzyloxy4-methoxy-benzylchlorotriphenylphosphate with 4-benzyloxybenzylbenzylchloride triphenyl phosphate to obtain 513 mg of yellow oily products 7e with a yield of 74.10%.

1H NMR (400 MHZ, CDCl₃) δ 7.15 (dt, J=7.4, 1.0 Hz, 1H), 7.09-7.03 (m, 1H), 6.48 (d, J=1.0 Hz, 1H), 3.82 (d, J=2.0 Hz, 4H), 2.89 (m, 2H); ¹³C NMR (101 MHz, CDCl₃) δ 163.64, 153.11, 153.09, 150.70, 150.68, 150.64, 150.62, 136.76, 136.71, 136.66, 136.37, 134.56, 130.45, 130.42, 130.40, 130.39, 130.37, 121.77, 121.76, 121.75, 121.74, 121.71, 121.70, 121.69, 121.68, 106.59, 106.56, 106.54, 106.51, 106.49, 63.07, 60.80, 56.12, 38.09, 38.07, 38.06, 38.03, 37.65, 37.63, 37.60.

### Example 6

### Preparing 5-(3,5-dimethoxyphenylethyl)-2-methoxyphenyl 2,2-dichloroacetate (7f)

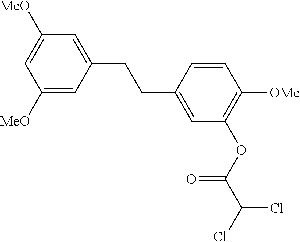 [SMILES: COc1cc(CCc2ccc(OC)c(OC(=O)C(Cl)Cl)c2)cc(OC)c1]

The synthesis method is as same as that of 7a, finally substitute 3,4,5-trimethoxybenzaldehyde with 3,4,5-trimethoxybenzaldehyde to obtain 557 mg of yellow oily products 7f with a yield of 80.45%.

1H NMR (400 MHZ, CDCl₃) δ 6.92 (m, 1H), 6.38 (m, 1H), 3.88 (s, 1H), 3.77 (s, 2H), 2.91 (m, 2H);¹³C NMR (101 MHz, CDCl₃) δ 163.83, 160.88 (dd, J=2.7, 1.4 Hz), 149.52 (dd, J=7.1, 2.6 Hz), 143.73 (ddd, J=5.0, 3.7, 1.8 Hz), 141.13 (dd, J=7.2, 1.0 Hz), 134.52 (dt, J=7.4, 1.7 Hz), 123.20 (dddt, J=9.0, 6.3, 3.2, 1.4 Hz), 120.37 (ddd, J=6.5, 3.6, 1.4 Hz), 112.56 (tt, J=7.4, 2.5 Hz), 107.29 (dt, J=6.0, 2.9 Hz), 98.06 (td, J=5.9, 2.4 Hz), 63.17, 56.00, 55.31, 37.41 (ddd, J=10.3, 4.6, 2.3 Hz).

### Example 7

Preparing 5-(3,4-dimethoxyphenylethyl)-2-methoxyphenyl 2,2-dichloroacetate (7 g).

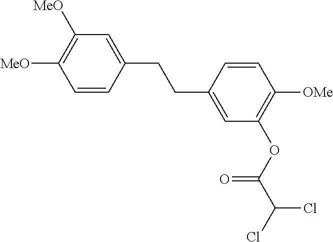 [SMILES: COc1ccc(CCc2ccc(OC)c(OC(=O)C(Cl)Cl)c2)cc1OC]

The synthesis method is as same as that of 7a, finally substitute 3,4,5-trimethoxybenzaldehyde, with 3,4-dimethoxybenzaldehyde to obtain 571 mg of yellow oily products 7 g with a yield of 82.47%.

1H NMR (400 MHZ, CDCl₃) δ 6.92 (m, 4H), 6.81 (m, 4H), 6.27 (s, 1H), 3.88 (s, 3H), 3.83 (d, J=1.3 Hz, 8H), 2.85 (d, J=1.5 Hz, 5H);¹³C NMR (101 MHz, CDCl₃) δ 163.83, 149.75, 149.73, 149.69, 149.54, 149.51, 147.86, 147.84, 141.50, 141.43, 135.16, 134.64, 134.58, 123.24, 123.22, 123.20, 123.18, 121.50, 121.48, 121.45, 120.41, 120.40, 120.38, 120.36, 120.35, 120.32, 112.62, 112.50, 112.10, 63.17, 56.00, 55.91, 55.88, 37.76, 37.69, 37.30.

### Example 8

### Preparing 2-methoxy-5-(3-methoxyphenyletyl)phenyl 2,2-dichloroacetate (7h)

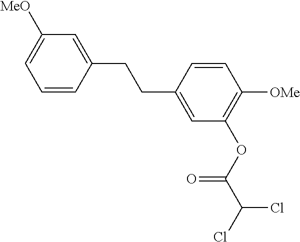 [SMILES: COc1cccc(CCc2ccc(OC)c(OC(=O)C(Cl)Cl)c2)c1]

The synthesis method is as same as that of 7a, finally substitute 3,4,5-trimethoxybenzaldehyde with 3-methoxybenzaldehyde to obtain 684 mg of yellow oily products 7h with a yield of 95.80%.

¹H NMR (400 MHZ, CDCl₃) δ 7.23 (m, 1H), 6.92 (m, 1H), 6.90 (m, 2H), 6.79 (m, 3H), 6.27 (s, 1H), 3.87 (s, 2H), 3.78 (s, 2H), 2.89 (m, 4H);¹³C NMR (101 MHz, CDCl₃) δ 163.86, 159.73, 159.68, 159.66, 149.67, 149.65, 143.39, 143.37, 143.36, 143.35, 143.34, 141.97 134.64, 134.62, 134.58, 129.43, 129.40, 129.36, 129.35, 129.34, 123.66, 123.58, 120.48, 120.47, 120.39, 115.09, 115.06, 115.04, 115.02, 114.98, 114.46, 114.34, 113.97, 113.83, 112.53, 112.49, 112.48, 112.45, 63.17, 56.00, 36.78, 36.32.

### Example 9

### Preparing 2-methoxy-5-(4-methoxyphenylethyl) phenyl 2,2-dichloroacetate (7i)

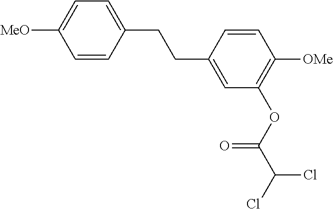 [SMILES: COc1ccc(CCc2ccc(OC)c(OC(=O)C(Cl)Cl)c2)cc1]

The synthesis method is as same as that of 7a, finally substitute 3,4,5-trimethoxybenzaldehyde with 3-methoxybenzaldehyde to obtain 659 mg of yellow oily products 7i with a yield of 92.20%.

¹H NMR (400 MHZ, CDCl₃) δ7.23 (m, 1H), 6.91 (m, 1H), 6.89 (m, 2H) 6.80 (m, 3H), 6.28 (s, 1H), 3.87 (s, 2H), 3.79 (s, 2H), 2.83 (s, 4H);¹³C NMR (101 MHz, CDCl₃) δ 163.86, 158.96, 158.88, 149.65, 149.59, 141.97, 141.96, 134.64, 134.45, 130.84, 130.78, 123.69, 123.60, 123.58, 120.07, 120.04, 119.98, 113.87, 112.53, 112.48, 112.46, 112.42, 63.17, 56.00, 55.32, 37.46, 37.44, 37.35, 37.28, 37.21.

### Example 10

### Preparing 2-methoxy-5-(3,4,5-trimethoxyphenylethyl) phenyl3-(2,2-dichloroacetamide) acetate (8a)

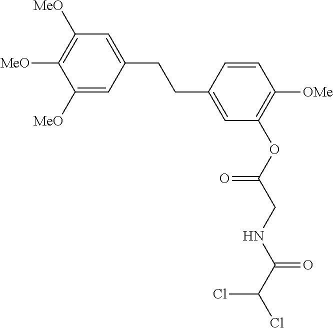 [SMILES: COc1ccc(CCc2cc(OC)c(OC)c(OC)c2)cc1OC(=O)CNC(=O)C(Cl)Cl]

Add 80 mL of dry methylene chloride and 8.3 mL (59.74 mmol) of triethylamine into 3 g (23.89 mmol) of methyl glycinate hydrochloride, then stirring the solution in an ice salt bath for 15 min. Slowly add dichloroacetyl chloride dropwise and continue reacting and stirring for 1.5 hours. After finishing the reaction, add 20 ml of water for quenching, then add 40 mL of dichloromethane to extract out an organic phase, and get an extract from the water layer by means of 20×2 dichloromethane. Merge the organic phases into one, then dry it by means of anhydrous sodium sulfate, next obtain a yellow oily product through reduced pressure distillation, finally obtain 3.98 g of compounds 11a through separation by flash paper chromatography. Add 3 g (15 mmol) of the compounds 11a into 30 mL of methanol and water (4:1), then add 718 mg (30 mmol) of lithium hydroxide and keeping reacting for 6 hours. After finishing the reaction, remove the solvent through reduced pressure distillation, then add 40 mL of water, and adjust the PH to 3-4 with 2M hydrochloric acid, then add 90 mL of ethyl acetate to extract out an organic phase, and get an extract from the water layer by means of 60×2 ethyl acetate. Merge the organic phases into one, then dry it by means of anhydrous sodium sulfate, next obtain a yellow oily product through reduced pressure distillation, finally obtain 1.6 g of compounds 6a through separation by flash paper chromatography. Add 150 mg (0.806 mmol) of the compounds 6a into 3 ml of dry methylene chloride, and add 368 mg (0.967 mmol) of HATU, stirring the solution in an ice salt bath for 10 min, then add 0.199 mL (1.21 mmol) of DIPEA, after stirring and reacting for 15 min, add 257 mg of erianin; after keeping reacting for 3 hours, filtered out a white solid, which is separated by flash column chromatography to obtain 254 mg of compound 8a with a yield of 64.79%.

¹H NMR (400 MHZ, CDCl₃) δ 7.00 (d, J=8.4 Hz, 1H), 6.89 (d, J=8.6 Hz, 2H), 6.34 (s, 2H), 6.02 (s, 1H), 4.39 (d, J=5.2 Hz, 2H), 3.81 (d, J=4.5 Hz, 12H), 2.84 (s, 4H); ¹³C NMR (101 MHz, CDCl₃) δ 167.30, 164.41, 153.09, 149.00, 138.89, 137.23, 136.16, 134.47, 127.33, 122.55, 112.40, 105.43, 66.00, 60.93, 56.10, 56.06, 41.84, 38.29, 36.99.

### Example 11

### Preparing 2-methoxy-5-(3,4,5-trimethoxyphenyletyl) phenyl3-(2,2-dichloroacetamide) propionate (8b)

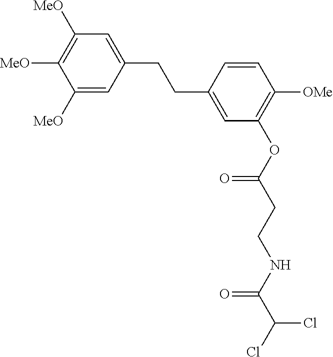 [SMILES: COc1ccc(CCc2cc(OC)c(OC)c(OC)c2)cc1OC(=O)CCNC(=O)C(Cl)Cl]

The synthesis method is as same as that of the compound 8a, finally substitute a methyl glycinate hydrochloride salt with a methyl alanine hydrochloride salt to obtain 294 mg of compounds 8b with a yield of 78.34%.

¹H NMR (400 MHZ, CDCl₃) δ 6.99 (dd, J=8.4, 2.2 Hz, 1H), 6.88 (d, J=8.4 Hz, 1H), 6.84 (d, J=2.1 Hz, 1H), 6.34 (s, 2H), 6.01 (s, 1H), 3.84-3.77 (m, 12H), 3.69 (q, J=6.1 Hz, 2H), 2.83 (s, 6H);¹³C NMR (101 MHZ, CDCl₃) δ 170.16, 164.42, 153.06, 149.05, 139.18, 137.30, 136.13, 134.40, 126.99, 122.75, 112.35, 105.55, 66.49, 60.86, 56.07, 38.20, 36.93, 36.03, 33.45.

### Example 12

### Preparing 2-methoxy-5-(3,4,5-trimethoxyphenylethy) phenyl4-(2,2-dichloroacetamide) butyrate (8c)

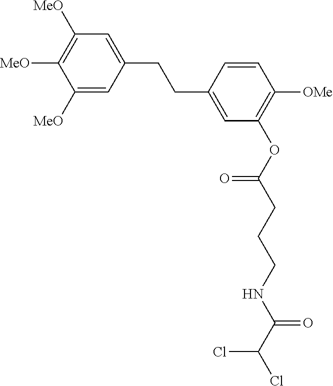 [SMILES: COc1ccc(CCc2cc(OC)c(OC)c(OC)c2)cc1OC(=O)CCCNC(=O)C(Cl)Cl]

The synthesis method is as same as that of the compound 8a, finally substitute a methyl glycinate hydrochloride salt with a γ-aminobutyric acid methyl hydrochloride salt to obtain 182 mg of the compound 8c with a yield of 50.48%.

¹H NMR (400 MHZ, CDCl₃) δ 7.46 (t, J=7.1 Hz, 1H), 6.94-6.88 (m, 1H), 6.92-6.83 (m, 2H), 6.51 (t, J=0.9 Hz, 2H), 6.28 (s, 1H), 3.87 (s, 2H), 3.82 (d, J=5.7 Hz, 9H), 3.26 (q, J=7.2 Hz, 2H), 2.87-2.79 (m, 4H), 2.43 (t, J=7.1 Hz, 2H), 1.78 (p, J=7.1 Hz, 2H); ¹³C NMR (400 MHZ, CDCl₃) δ 171.52, 168.36, 153.29, 149.60, 140.73, 136.79, 134.51, 134.48, 123.48, 123.41, 120.76, 112.50, 105.89, 60.75, 56.13, 40.17, 36.63, 32.44, 32.41, 24.90, 24.81.

### Example 13

### Preparing 2-methoxy-5-(3,4,5-trimethoxyphenylethyl) phenyl6-(2,2-dichloroacetamide) hexanoate (8d)

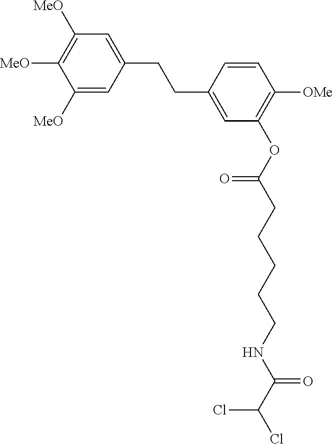 [SMILES: COc1ccc(CCc2cc(OC)c(OC)c(OC)c2)cc1OC(=O)CCCCCNC(=O)C(Cl)Cl]

The synthesis method is as same as that of the compound 8a, finally substitute a methyl glycinate hydrochloride salt with a methyl aminocaproic acid hydrochloride salt to obtain 227 mg of compounds 8d with a yield of 67.54%.

¹H NMR (400 MHZ, CDCl₃) δ 6.97 (dd, J=8.3, 2.1 Hz, 1H), 6.90-6.81 (m, 3H), 6.35 (s, 2H), 5.95 (s, 1H), 3.83-3.78 (m, 12H), 3.34 (q, J=6.7 Hz, 2H), 2.83 (s, 4H), 2.58 (t, J=7.3 Hz, 2H), 1.78 (p, J=7.4 Hz, 2H), 1.63 (p, J=7.2 Hz, 2H), 1.47 (tt, J=9.6, 5.9 Hz, 2H); ¹³C NMR (101 MHz, CDCl₃) δ 171.76, 164.31, 153.05, 149.32, 139.51, 137.46, 134.32, 126.74, 122.86, 112.35, 105.43, 66.61, 60.93, 56.08, 56.05, 40.19, 38.31, 37.02, 33.79, 28.74, 26.10, 24.51.

### Example 14

### In-Vitro Inhibitory Activity Against Proliferation of Tumor Cells

Take cells in logarithmic growth phase for experiments. Digest and count the cells, then make them into a cell suspension in 1×10⁵ cells/mL, next seed the cell suspension in a 96-hole plate (100 μL per hole), then place the plate in a 5% CO₂ incubator at 37° C. to be incubated for 24 hours. Add a subject with a corresponding concentration into each hole, and concurrently set up a negative control group and a blank group, each of which has 3 holes. After having placed the plate in the incubator for 72 hours, observe the morphologies of cells in each group under a microscope, then add a 10 μL CCK8 solution to each hole, and continue incubating the cells in the incubator for 4 h. Measure the absorbance value at 450 nm, and calculate the inhibition rate of cell proliferation; if P<0.05, the results have statistical significance. The results are shown in Table 1.

### 2. Test Results

The results showed that the compounds 7a and 8d have good inhibitory activity against HepG2 cells, and the inhibition rate of the compound 7b against the HepG2 is better than that of erianin.

TABLE 1
In vitro activity evaluation of some exampled compounds against
the HepG2 cell strain.

| | Inhibition rate(%) | | |
|---|---|---|---|
| CompoundsNo. | 10 μM | 1 μM | 0.1 μM |
| erianin | 95.05 ± 0.85** | 97.15 ± 1.62 | 21.68 ± 9.14 |
| 7a | 89.27 ± 1.50 | 94.43 ± 0.89 | / |
| 7b | 73.7 ± 4.70** | 48.4 ± 9.88** | 35.71 ± 2.42** |
| 8a | 45.62 ± 2.53** | 29.99 ± 2.03 | 18.46 ± 1.16 |
| 8b | 74.17 ± 2.41** | 55.84 ± 4.37** | 37.48 ± 4.49** |
| 8c | 89.27 ± 0.66** | 68.68 ± 0.72** | 36.06 ± 11.67** |
| 8d | 90.57 ± 2.33** | 72.97 ± 3.10** | 50.94 ± 4.44** |

Where, *P < 0.05, **p < 0.01.

### Example 15

Adopting the CCK-8 method to test the anti-tumor activity of the compound against a variety of tumor cells.

### 1. Test Method

Take cells in logarithmic growth phase for experiments. Digest and count the cells, then make them into a cell suspension in 1×10⁵ cells/mL, next seed the cell suspension in a 96-hole plate (100 μL per hole), then place the plate in a 5% CO₂ incubator at 37° C. to be incubated for 24 hours. Add a subject with a corresponding concentration into each hole, and concurrently set up a negative control group and a blank group, each of which has 3 holes. After having placed the plate in the incubator for 72 hours, observe the morphologies of cells in each group under a microscope, then add a 10 μL CCK8 solution to each hole, and continue incubating the cells in the incubator for 4 hours, measure the absorbance value at 450 nm. In the test, take cells with 8 concentration gradients respectively, then measure the inhibition rate corresponding to each concentration. Use the GraphPad Prism 5.0 statistical software to treat data, use the t-test software to make a comparison between two groups, and use the One-way Anova (Dunnett) software to make a comparison among multiple groups. if P<0.05, the results have statistical significance, and then calculate the IC₅₀ value. The results are shown in Table 2.

### 2. Test Results

TABLE 2
In vitro activity evaluation of some exampled compounds against
multiple tumor cell strains.

| | IC₅₀(μM) | | |
|---|---|---|---|
| cell strain | compound 7a | compound 8d | erianin |
| SGC7901 | 0.4690 | 0.1031 | 0.1759 |
| A549 | 0.7261 | 02162 | 0.0526 |
| Hela | 0.5160 | 0.2883 | 8.300 |
| MCF-7 | 0.6555 | 0.1736 | 0.1959 |
| HepG2 | 0.0489 | 0.1351 | 0.0728 |
| SW480 | 0.9403 | 0.3261 | 0.0245 |

The results of the above anti-tumor activity evaluation of tumor cells cultured in vitro showed that the dichloroacetic acid conjugating diphenyl ethane compound has the inhibitory activity widely against various tumor cell strains such as human gastric cancer SGC7901 cell strains, human lung adenocarcinoma A549 cell strains, human cervical cancer Hela cell strains, human breast cancer MCF-7 cell strains, human hepatoma HepG2 cell strains and human colon cancer SW480 cell strains. The compound has a IC₅₀ value against the Hela cells as 0.5160 μM/mL, which is considerably different in activity from the IC₅₀ value of erianin as 8.3 μM/mL, indicating that the introduction of dichloroacetic acid in the compound 7a based on erianin greatly improves the inhibitory activity of the compound against the Hela cells.

Form comparison from in vitro anti-tumor activity, the compound 7a is more selective in the aspect of the anti-tumor activity against HepG2 hepatoma cell strains, with an IC₅₀ value against the HepG2 cells as 0.0489 μM/mL, nearly doubling its activity with respect to the IC 50 value of erianin, 0.0728 μM/mL.

The compound 8d has inhibitory activity superior to erianin against human gastric cancer SGC7901 cell strains and human breast cancer MCF-7 cell strains. The compound has a IC₅₀ value against the Hela cells as 0.2883 μM/mL, while the erianin has a IC 50 value as 0.0728 μM/mL, thus the inhibitory effect of the compound 8d against the Hela cells is also significantly better than that of mullan.

The above results show that the dichloroacetic acid conjugating diphenyl ethane compound of the present invention has high anti-tumor activity, which is significantly better than that of erianin in a positive control group, so it has a good application and development prospect.

### Example 16

An acute toxicity test of single gavage administration to mice.

### 1. Test Method

Choose ICR mice (SPF grade) as subjects, and keep them fasted overnight before gavage. Randomly divide 20 mice (weigh: 19-22 g, half male and half female) administrated at a dosage of 5000 mg/kg·bw into groups according to their weighs. Take a single gavage administration at the maximum dosage of the tested drug as 5000 mg/kg, observe the mice once respectively in 0.5h, 1h, 2h, 4h and 24h, and record the mortality rate, then keep observing them once every day and recording the mortality rate for 154 days. On the 15th day, kill an undead mouse and perform pathological dissection.

### 2. Test Results

As for the single gavage administration, there is no animal death (LD>5000 mg/kg) at a high, medium and low dosage in the form of oral administrations, no obvious residual drug liquid is found during the dissection, and no abnormalities occurs in the heart, lung, spleen, kidney and other organs during the dissection, indicating that the safety of the tested drug is good.

### Example 17

A test for the gavage administration on the rate of inhibiting xenografted tumors of tumor-bearing nude mice.

### 1. Test Method

Inoculate the human hepatoma cell strain HepG2 subcutaneously at an armpit of a nude mouse, as to establish a human hepatoma cell HepG2 xenotransplantation tumor model. Take the cells HepG2 in logarithmic growth phase, under sterile conditions, inoculate the cells subcutaneously at right armpits of 55 nude mice. Measure the diameter of the xenograft tumors by a vernier caliper, when the tumors grow to about 100 mm³, choose 48 tumor-bearing nude mice having an uniform tumor size in a good growth status, then randomly divide them into 8 groups, each of which has 6 mice, namely a model group, a 7a low-dosage group (50 mg/kg), a 7a medium-dosage group (100 mg/kg), a 7a high-dosage group (150 mg/kg), and a sorafenib group (50 mg/kg). Share an isovolumic solvent among the mice in the model group through gavage, administer drugs through gavage once a day. Administer drugs in the 7a low-dosage group, the 7a medium-dosage group, and the 7a high-dosage group once a day at 50 mg/kg, 100 mg/kg, and 150 mg/kg, respectively, to reach 14 times administration in total. Administer drugs in the sorafenib group once a day at 50 mg/kg, to reach 14 times administration in total. Dynamically observe the anti-tumor effect of the subjects by adopting the method of measuring tumor diameters. Measure the tumor diameter on alternate days, while weigh the body weight of nude mice. On the 15^{th} day, kill the mice, then pick away tumor lumps surgically and weigh them, and preserve the tumor tissue in a liquid nitrogen tank, next calculate the tumor inhibition rate (%). Tumor inhibition rate (%)= (tumor weight in the model group-tumor weight in the administration group)/tumor weight in the model group×100%.

### 2. Test Results

According to the administration plan, the aforementioned compound can significantly inhibit the growth of the xenografted tumors of tumor-bearing nude mice, it can be observed that the tumors trend to shrink on about the 11^{th} day in the low-dosage group after administration; it can be observed that the tumors trend to shrink on about the 9^{th} day in the medium-dosage group after administration; it can be observed that the tumors trend to obviously shrink on about the 7^{th} day in the high-dosage group after administration.

TABLE 3
Inhibition effect against the growth of tumors of human
hepatocellular carcinoma cells HepG2 xenografted to nude mice.

| | | Number | Number | | |
|---|---|---|---|---|---|
| | Dosage | of initial | of final | Tumor | Inhibition |
| Group | (mg/kg) | mice | mice | weight (g) | rate(%) |
| 7a low-dosage | 50 | 6 | 6 | 0.569 ± 0.103** | 62.0 |
| group | | | | | |
| 7a medium- | 100 | 6 | 6 | 0.479 ± 0.100** | 72.4 |
| dosage group | | | | | |
| 7a high-dosage | 150 | 6 | 6 | 0.314 ± 0.076* | 86.9 |
| group | | | | | |
| sorafenib group | 50 | 6 | 6 | 0.581 ± 0.089** | 59.3 |

The above results show that the compound of the present invention can significantly inhibit the growth of tumor in vivo and the tumor trends to shrink, and with the increase of the dosage of compound, the speed of tumor shrinkage increases. Sorafenib is a multi-targeted anti-tumor oral drug widely used in clinical practice, while the compound of the present invention has a tumor inhibition rate better than that of sorafenib, and has better safety (LD50>5000 mg/kg).

The above relevant description and explanation to the examples are intended to help a person skilled in the art to understand and apply the present invention. It is clear that a person skilled in the art can easily make various modifications to these contents and apply the general principles described herein to other embodiments without doing creative work. Therefore, the present invention is not limited to the above relevant description and explanation to the examples, and the improvements and modifications made by a person skilled in the art according to the disclosure of the present invention and not departing from the scope of the present invention should fall within the protection scope of the present invention.

What is claimed:

1. A dichloroacetic acid conjugating diphenyl ethane compound, comprising a structure having a general formula (I): 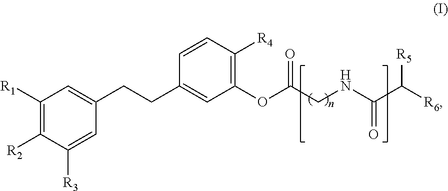
where, R₁, R₂ and R₃ are respectively selected from, but not limited to, any one of —OH, —Ome and H, R₄ is selected from, but not limited to, any one of —OMe and —OEt, R₅ and R₆ are respectively selected from any one of a chlorine atom and a fluorine atom, m is 0 or 1, and n is selected from any one of 1, 2, 3, 4, 5, 6, 7, 8, 11 and 12.

2. The dichloroacetic acid conjugating diphenyl ethane compound of claim 1, wherein the compound is selected from any one of:

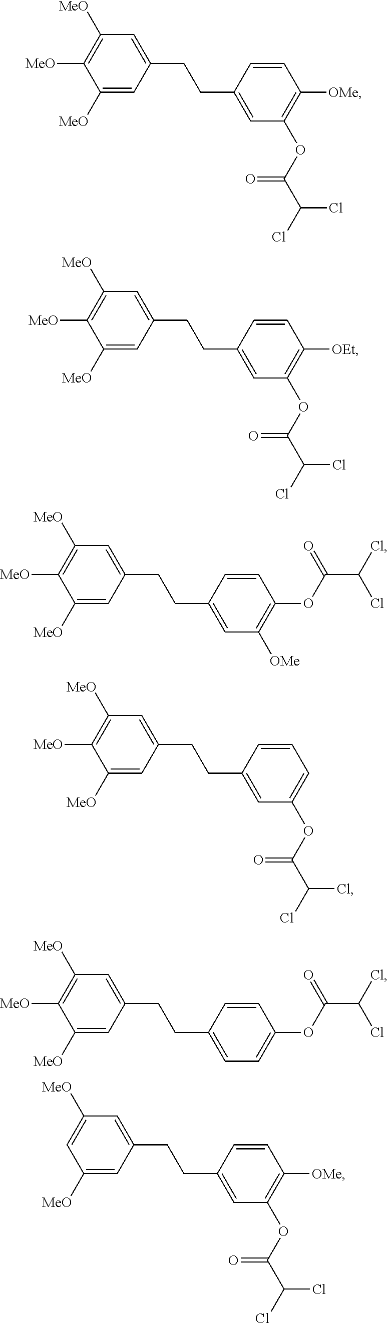

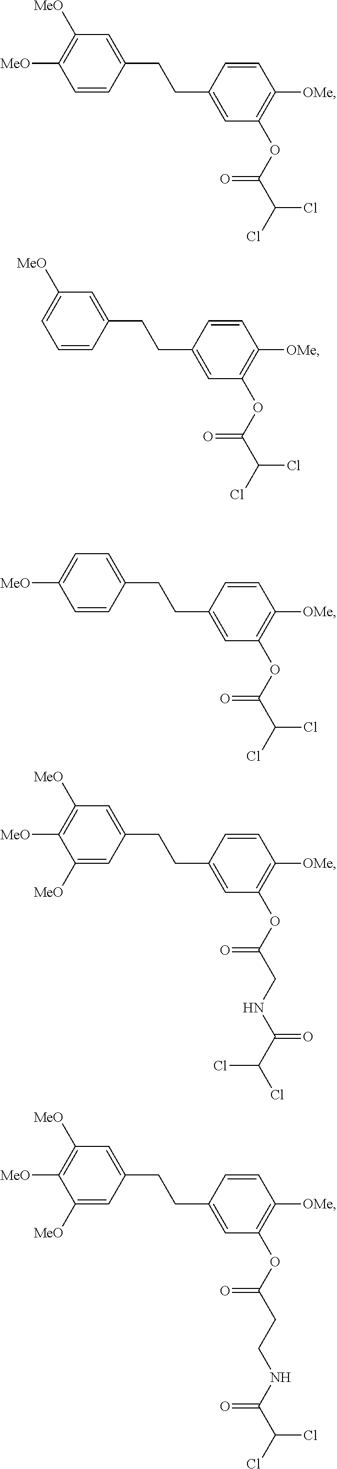 [SMILES: COc1ccc(CCc2ccc(OC)c(OC(=O)C(Cl)Cl)c2)cc1OC ; COc1cccc(CCc2ccc(OC)c(OC(=O)C(Cl)Cl)c2)c1 ; COc1ccc(CCc2ccc(OC)c(OC(=O)C(Cl)Cl)c2)cc1 ; COc1ccc(CCc2cc(OC)c(OC)c(OC)c2)cc1OC(=O)CNC(=O)C(Cl)Cl ; COc1ccc(CCc2cc(OC)c(OC)c(OC)c2)cc1OC(=O)CCNC(=O)C(Cl)Cl]

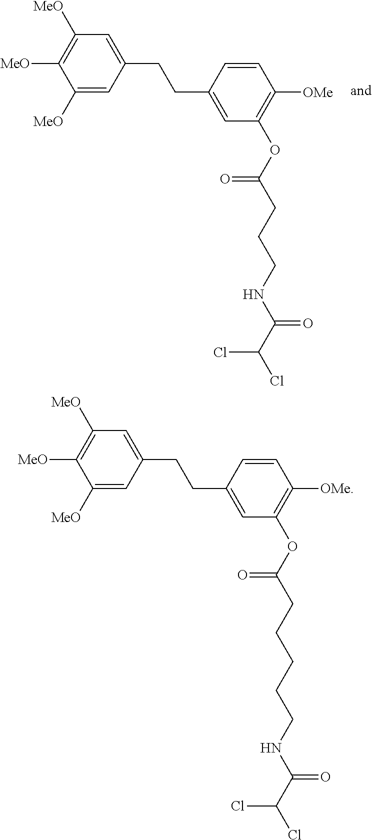 [SMILES: COc1ccc(CCc2cc(OC)c(OC)c(OC)c2)cc1OC(=O)CCCNC(=O)C(Cl)Cl ; COc1ccc(CCc2cc(OC)c(OC)c(OC)c2)cc1OC(=O)CCCCCNC(=O)C(Cl)Cl]

3. A pharmaceutical composition comprising an active ingredient comprising the dichloroacetic acid conjugating diphenyl ethane compound of claim 1.

4. The pharmaceutical composition of claim 3, wherein the composition further comprises a pharmaceutically acceptable carrier.

5. The pharmaceutical composition of claim 3, wherein the compound has a pharmaceutical form selected from, but not limited to, lyophilized powders, powders, injections, liposomes, emulsions, microcapsules, suspensions or solutions, whichever are administered intravenously; granules, tablets, capsules, or syrups, whichever are administered orally; or suppositories.

6. The pharmaceutical composition of claim 3, wherein the compound comprises a drug that inhibits proliferation, growth or damage of tumor cells, and eliminates tumor cells.

7. A method of preparing the dichloroacetic acid conjugating diphenyl ethane compound of claim 1 at the existence of —OH in place of more than one of R₁, R₂ and R₃, comprising the steps of:
(a) protecting a hydroxyl group in 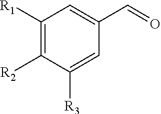
with benzyl chloride to obtain a corresponding benzyl-protected product;
(b) making the benzyl-protected product and a triphenyl phosphorus ylide 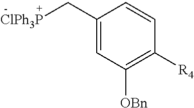
execute a Wittig reaction to obtain a benzyl-protected stilbene compound;
(c) reducing and deprotecting the benzyl-protected stilbene compound to obtain a corresponding diphenyl ethane compound; and
(d) at the existence of m=0, making 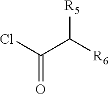
combine with the corresponding diphenyl ethane compound through a condensation reaction to obtain a dichloroacetic acid conjugating diphenyl ethane compound or a dichloroacetic acid conjugating stilbene compound.

8. The method of claim 7, wherein, in the absence of —OH in place of any one of R₁, R₂ and R₃, the step of protection with benzyl chloride is omitted, 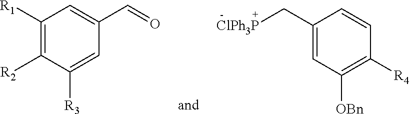
directly execute a Wittig reaction.

9. The preparation method of claim 7, wherein the dichloroacetic acid is combined with the diphenyl ethane by way of an amino acid having a chain different in length, and the method further includes the steps of:
(e) making an amino acid having a chain different in length 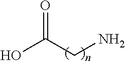
react with thionyl chloride to obtain a corresponding amino acid methyl ester hydrochloride 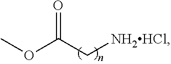
where, n=1, 2, 3, 4, 6, 7, 8, 11 or 12;
(f) combining the amino acid methyl ester hydrochloride having a chain different in length with 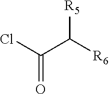
through a condensation reaction to obtain a compound 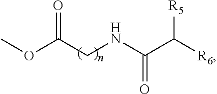
where n=1, 2, 3, 4, 6, 7, 8, 11 or 12;
(g) hydrolyzing and demethylating the compound 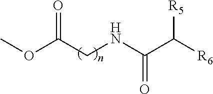
to obtain a corresponding acid 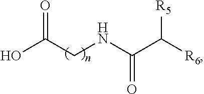
where n=1, 2, 3, 4, 6, 7, 8, 11 or 12; and
(h) combining 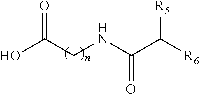
with the corresponding diphenyl ethane compound through a condensation reaction to obtain a corresponding dichloroacetic acid conjugating diphenyl ethane compound (I), where n=1,2,3,4,6,7,8,11 or 12.

10. A method comprising manufacturing an anti-tumor drug comprising the dichloroacetic acid conjugating diphenyl ethane compound of claim 1.

11. The method of claim 10, wherein the anti-tumor drug resists tumors or neoplasms derived from one or more of a gastric cancer cell, a lung cancer cell, a cervical cancer cell, a bowel cancer cell, a breast cancer cell and a liver cancer cell.

12. The method of claim 10, wherein the anti-tumor drug includes a drug or a reagent that alleviates or inhibits tumor cell proliferation.

13. The method of claim 10, wherein concentrations of the dichloroacetic acid conjugating diphenyl ethane compound in a cell culture medium range from 0.1 to 10 μM.

14. A reagent for inhibiting proliferation of a cell, comprising the dichloroacetic acid conjugating diphenyl ethane compound of claim 1.

15. The reagent for inhibiting proliferation of a cell of claim 14, wherein the cell is a tumor cell cultured in vitro.
